(19) United States
(12) Patent Application Publication
(10) Pub. No.: US 2024/0138821 A1
(43) Pub. Date: May 2, 2024

(54) SLOTTED SPECIMEN HOLDER, WIRELESS TRANSPONDER LOADING CARTRIDGE, WIRELESS TRANSPONDER DISPENSER AND METHODS

(71) Applicant: TMRW Life Sciences, Inc., New York, NY (US)

(72) Inventors: James Craven, Ely (GB); Wayne Bennett, Bedford (GB)

(21) Appl. No.: 18/385,232

(22) Filed: Oct. 30, 2023

Related U.S. Application Data
(60) Provisional application No. 63/421,490, filed on Nov. 1, 2022.

(51) Int. Cl. A61B 10/00; A61B 10/02; A61B 90/98

(52) U.S. Cl. CPC A61B 10/0096; A61B 10/0291; A61B 90/98

(57) ABSTRACT

A specimen holder includes a body, a cavity that extends into the body through an opening, and at least one slot. The specimen holder further includes a wireless transponder insertable into the cavity by increasing a dimension of the cavity, by increasing a dimension of the at least one slot. A wireless transponder loading cartridge includes a cavity that extends from an opening in the cartridge, and a plurality of wireless transponders positioned within the cavity. Each of the plurality of wireless transponders includes an outer peripheral shape that corresponds to a cross-sectional shape of the cavity. A wireless transponder dispenser includes a housing with a first recess that receives the cartridge and a second recess that receives the specimen holder. The wireless transponder dispenser provides a passage for wireless transponders from the cavity of the cartridge to a loading zone aligned with the cavity of the specimen holder.

### BACKGROUND

### Technical Field

The present disclosure relates generally to articles, systems and methods of specimen collection, and specifically to an article, system and components thereof, and method that enables identification of collected specimen, for example during patient procedures, including but not limited to diagnostics, treatment, human fertility (such as an IVF procedure), biobanking, etc.

### Description of the Related Art

Long-term preservation of cells and tissues through cryopreservation has broad impacts in multiple fields including tissue engineering, fertility and reproductive medicine, regenerative medicine, stem cells, blood banking, animal strain preservation, clinical sample storage, transplantation medicine, and in vitro drug testing. This can include the process of vitrification in which a biological sample (e.g., an oocyte, an embryo, a biopsy) contained in or on a storage device (e.g., a cryopreservation straw, cryopreservation tube, stick or spatula) is rapidly cooled by placing the biological sample and the storage device in a substance, such as liquid nitrogen.

This results in a glass-like solidification or glassy state of the biological sample (e.g., a glass structure at the molecular level), which maintains the absence of intracellular and extracellular ice (e.g., reducing cell damage and/or death) and, upon thawing, improves post-thaw cell viability. To ensure viability, the vitrified biological samples are then typically continuously stored in a liquid nitrogen dewar or other container, which is at a temperature conducive to cryopreservation, for example negative 196 degrees Celsius.

There are, however, a number of concerns in how these biological samples are being stored, identified, managed, inventoried, retrieved, etc. For example, each harvested embryo is loaded on a rigid embryo straw, tube, stick or spatula. The tube may be open at one end that receives the harvested embryo and closed (e.g., plugged) at the other end. The cryopreservation storage devices containing or holding the embryos are cooled as quickly as possible by plunging the cryopreservation storage device with the biological material into liquid nitrogen at a temperature of approximately negative 196 degrees Celsius, for example to achieve vitrification.

More particularly, multiple cryopreservation storage devices are placed in a goblet for placement in the liquid nitrogen storage tank. The goblet attaches to the liquid nitrogen storage tank such that the multiple cryopreservation storage devices are suspended in the liquid nitrogen. Labels that are manually written-on using a suitable marker pen or printed using a custom printer are attached to the straw and/or the goblet. Such labels can include identification information corresponding to the individual that the embryo was harvested from and other suitable information (e.g., a cryopreservation storage device number, a practitioner number, etc.).

Stored biological samples can be identified by writing on the storage devices themselves, or by labels stuck to the storage devices. These labels may be handwritten or printed and can include bar codes. However, such methods of identification have associated disadvantages. Written notes on containers can be erased or smudged and labels can fall off the storage devices while they are stored inside the dewar leading to unidentifiable samples. These problems are exacerbated by the cold conditions in which biological samples are kept.

When performing an audit of biological samples stored in cold storage (typically at temperatures of negative 196 degrees Celsius), warming of the samples to a temperature greater than negative 130 degrees Celsius is to be avoided. It is therefore desirable to minimize the amount of time spent outside of the dewar wherever possible.

Recording, monitoring and auditing of samples in cold storage takes a considerable amount of time and effort, even when samples are labelled using barcodes. An additional and undesirable increase in the time taken to record or audit samples arises as a result of frost which can form on the surfaces of the storage devices and their labels when they are removed from liquid nitrogen into relatively warmer temperatures. A layer of frost blocks optical observance of the identification information, and the layer of frost also diffracts the light of a bar code reader. The container cannot be warmed up to remove frost as this would lead to destruction of the sample. The frost can be wiped off the disposable container, but this contributes to an undesirable increase in the amount of time taken to read the sample.

Accordingly, it is desirable to provide a new apparatus for collecting, preserving, and identifying biological samples (e.g., vitrified biological samples) at suitably cold temperatures.

### BRIEF SUMMARY

According to one aspect of the disclosure, a specimen holder includes a body, a cavity, and at least one slot. The body is elongated along a body longitudinal axis, and the body has a distal end and a proximal end. The proximal end is opposite the distal end with respect to the body longitudinal axis. The body has a maximum length measured from the proximal end to the distal end along a first direction that is parallel to the longitudinal axis The body includes a distal portion that includes the distal end, and the body includes a proximal portion that includes the proximal end. The distal portion includes a surface that carries a specimen upon engagement of the body with the specimen. The cavity extends into the proximal portion from an opening formed in the proximal end, and the cavity terminates within the proximal portion. At least a portion of the cavity is formed by an inner surface of the proximal portion. The at least one slot extends from the proximal end in the first direction, and the at least one slot extends from an outer surface of the proximal portion to the inner surface.

According to one aspect of the disclosure, a method of assembling a specimen holder includes positioning a wireless transponder adjacent a distal end of a body. The body is elongated along a body longitudinal axis, and the body has a distal end and a proximal end opposite the distal end with respect to the body longitudinal axis. The method further includes inserting the wireless transponder through an opening formed in the proximal end and into a cavity that extends into the proximal portion from the opening in the proximal end.

The method further includes, while inserting the wireless transponder, increasing a cross-sectional dimension of the cavity that is measured in a direction perpendicular to the body longitudinal axis, and while inserting the wireless transponder, abutting both a first portion of the proximal portion and a second portion of the proximal portion with the wireless transponder. The first portion and the second portion are separated by at least one slot of the body, and the slot includes a length that extends from the proximal end in a direction parallel to the body longitudinal axis. The at least one slot further includes a depth that extends from an inner surface of the proximal portion that forms the cavity to an outer surface of the proximal portion that faces away from the cavity.

Abutting both the first portion of the proximal portion and the second portion of the proximal portion with the wireless transponder increases a width of the at least one slot. The width extends from a first side surface of the first portion to a second side surface of the second portion. The first and second side surfaces each extends between the inner surface and the outer surface of the proximal portion.

According to one aspect of the disclosure, a method of collecting a biological specimen includes positioning a biological specimen on a surface of a body that is elongated along a body longitudinal axis. The body has a distal end and a proximal end opposite one another with respect to the body longitudinal axis. The biological specimen is positioned closer to the distal end than the biological specimen is positioned from the proximal end.

The method further includes inserting a wireless transponder through an opening formed in the proximal end and into a cavity that extends into the body from the opening. While inserting the wireless transponder, a cross-sectional dimension of the cavity that is measured in a direction perpendicular to the body longitudinal axis is increased. While inserting the wireless transponder, both a first portion of the proximal portion and a second portion of the proximal portion are abutted with the wireless transponder. The first portion and the second portion are separated by at least one slot of the body. The slot includes a length that extends from the proximal end in a direction parallel to the body longitudinal axis, and the at least one slot further includes a depth that extends from an inner surface of the proximal portion that forms the cavity to an outer surface of the proximal portion that faces away from the cavity.

The method further includes abutting both the first portion of the proximal portion and the second portion of the proximal portion with the wireless transponder thereby increasing a width of the at least one slot. The width extends from a first side surface of the first portion to a second side surface of the second portion, and the first and second side surfaces each extend between the inner surface and the outer surface of the proximal portion.

According to one aspect of the disclosure, a wireless transponder dispenser includes a cartridge and a plurality of wireless transponders. The cartridge includes a first end and a second end, and the cartridge elongate along a direction. The cartridge has a first length measured along the direction from the first end to the second end, and the cartridge includes a cavity that extends from an opening formed by the first end towards the second end. The cavity includes a second length measured along the direction from the opening to a base surface of the cartridge. The base surface is opposite the second end, and the base surface is positioned closer to the second end than the base surface is from the first end.

The plurality of wireless transponders are positioned within the cavity, and each of the plurality of wireless transponders includes an outer peripheral shape that corresponds to a cross-sectional shape of the cavity. The cross-sectional shape lies entirely within a plane perpendicular to the direction, and the outer peripheral shape corresponds to the cross-sectional shape such that relative movement of the plurality of wireless transponders and the cartridge is constrained in all degrees of freedom other than translation along the direction.

According to one aspect of the disclosure, a method of dispensing a plurality of wireless transponders includes positioning a cartridge within a recess of a housing such that a cartridge cavity that extends from an opening formed by a first end of the cartridge toward a second end of the cartridge is aligned with a housing cavity that extends into the housing and away from the cartridge. The method further includes, after the cartridge is positioned within the recess, moving a wireless transponder from the cartridge cavity, through the opening, and into the housing cavity. The method further includes aligning the wireless transponder with an opening in the housing that provides passage into the housing cavity, and inserting a portion of a specimen holder through the opening in the housing and into the housing cavity such that the wireless transponder is aligned with a specimen holder cavity formed in the portion of the specimen holder, and translating the specimen holder relative to the wireless transponder thereby inserting the wireless transponder through an opening in the specimen holder and into the specimen holder cavity.

### BRIEF DESCRIPTION OF THE SEVERAL VIEWS OF THE DRAWINGS

In the drawings, identical reference numbers identify similar elements or acts. The sizes and relative positions of elements in the drawings are not necessarily drawn to scale. For example, the shapes of various elements and angles are not necessarily drawn to scale, and some of these elements may be arbitrarily enlarged and positioned to improve drawing legibility. Further, the particular shapes of the elements as drawn, are not necessarily intended to convey any information regarding the actual shape of the particular elements, and may have been solely selected for ease of recognition in the drawings.

FIG. 1 is an isometric view of a specimen holder, according to an aspect of the disclosure.

FIG. 2 is a side elevation view of the specimen holder illustrated in FIG. 1.

FIG. 3 is a top plan view of the specimen holder illustrated in FIG. 1.

FIG. 4 is a rear elevation view of the specimen holder illustrated in FIG. 1.

FIG. 5 is a rear elevation view of the specimen holder illustrated in FIG. 1, according to an aspect of the disclosure.

FIG. 6 is a rear elevation view of the specimen holder illustrated in FIG. 1, according to an aspect of the disclosure.

FIG. 7 is a rear elevation view of the specimen holder illustrated in FIG. 1, according to an aspect of the disclosure.

FIG. 8 is a rear elevation view of the specimen holder illustrated in FIG. 1, according to an aspect of the disclosure.

FIG. 9 is a rear elevation view of the specimen holder illustrated in FIG. 1, according to an aspect of the disclosure.

FIG. 10 is a front elevation view of the specimen holder illustrated in FIG. 1, according to an aspect of the disclosure.

FIG. 11 is a cross-sectional side view of the specimen holder illustrated in FIG. 1, along line A-A.

FIG. 12 is a cross-sectional top view of the specimen holder illustrated in FIG. 1, along line B-B.

FIG. 13 is a top plan view of a proximal portion of the specimen holder illustrated in FIG. 1, according to an aspect of the disclosure.

FIG. 14 is a top plan view of a proximal portion of the specimen holder illustrated in FIG. 1, according to an aspect of the disclosure.

FIG. 15 is a top plan view of a proximal portion of the specimen holder illustrated in FIG. 1, according to an aspect of the disclosure.

FIG. 16 is a cross-sectional top plan view of a proximal portion of the specimen holder illustrated in FIG. 1, according to an aspect of the disclosure.

FIG. 17 is a top plan view of a wireless transponder, according to an aspect of the disclosure.

FIG. 18 is a front elevation view of the wireless transponder illustrated in FIG. 17, according to an aspect of the disclosure.

FIG. 19 is a cross-sectional side view of the specimen holder illustrated in FIG. 1, along line A-A, with the wireless transponder illustrated in FIG. 17 positioned within a cavity of the specimen holder.

FIG. 20 is a cross-sectional side view of the specimen holder illustrated in FIG. 1, along line B-B, with the wireless transponder illustrated in FIG. 17 positioned within the cavity of the specimen holder.

FIG. 21 is a front, top, isometric view of a wireless transponder loading cartridge, according to an aspect of the disclosure.

FIG. 22 is a cross-sectional view of the cartridge illustrated in FIG. 21, along line C-C, with a plurality of wireless transponders positioned within a cavity of the cartridge.

FIG. 23 is a top plan view of the cartridge illustrated in FIG. 21.

FIG. 24 is a cross-sectional view of the cartridge illustrated in FIG. 21, along line C-C, with a plurality of wireless transponders positioned within a cavity of the cartridge, the cartridge including an actuator according to an aspect of the disclosure.

FIG. 25 is a cross-sectional view of the cartridge illustrated in FIG. 21, along line C-C, with a plurality of wireless transponders positioned within a cavity of the cartridge, the cartridge including an actuator according to an aspect of the disclosure.

FIG. 26 is another cross-sectional view of cartridge illustrated in FIG. 21, the cartridge including a stopper.

FIG. 27 is an isometric view of a wireless transponder dispenser according to an aspect of the disclosure.

FIG. 28 is a cross-sectional side view of the wireless transponder dispenser during use along line D-D, with a cartridge carrying a plurality of wireless transponders being positioned within a recess of the housing.

FIG. 29 is a cross-sectional side view of the wireless transponder dispenser during use along line D-D, with the cartridge carrying the plurality of wireless transponders positioned within the recess of the housing.

FIG. 30 is a cross-sectional side view of the wireless transponder dispenser during use along line D-D, with one of the plurality of wireless transponders aligned with a cavity of the specimen holder illustrated in FIG. 1.

FIG. 31 is a cross-sectional side view of the wireless transponder dispenser during use along line D-D, with one of the plurality of wireless transponders positioned within the cavity of the specimen holder.

FIG. 32 is a cross-sectional side view of the wireless transponder dispenser during use along line D-D, with the specimen holder and wireless transponder positioned adjacent an optical scanner of the wireless transponder dispenser.

### DETAILED DESCRIPTION

In the following description, certain specific details are set forth in order to provide a thorough understanding of various disclosed embodiments. However, one skilled in the relevant art will recognize that embodiments may be practiced without one or more of these specific details, or with other methods, components, materials, etc. In other instances, well-known structures associated with specimen collection systems have not been shown or described in detail to avoid unnecessarily obscuring descriptions of the embodiments.

Unless the context requires otherwise, throughout the specification and claims which follow, the word “comprise” and variations thereof, such as, “comprises” and “comprising” are to be construed in an open, inclusive sense, that is as “including, but not limited to.”

Reference throughout this specification to “one embodiment” or “an embodiment” means that a particular feature, structure or characteristic described in connection with the embodiment is included in at least one embodiment. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

As used in this specification and the appended claims, the singular forms “a,” “an,” and “the” include plural referents unless the content clearly dictates otherwise. It should also be noted that the term “or” is generally employed in its broadest sense, that is as meaning “and/or” unless the content clearly dictates otherwise.

Reference herein to two elements “facing” or “facing toward” each other indicates that a straight line can be drawn from one of the elements to the other of the elements without contacting an intervening solid structure. Reference herein to two elements being “directly coupled” indicates that the two elements physically touch with no intervening structure between. Reference herein to a direction includes two components that make up said direction. For example, a longitudinal direction, which is bidirectional, includes both a “distal” component (unidirectional) and a “proximal” component (unidirectional), which is opposite the “distal” component. Reference to an element extending along a direction means the element extends along one or both of the components that make up the direction.

The term “aligned” as used herein in reference to two elements along a direction means a straight line that passes through one of the elements and that is parallel to the direction will also pass through the other of the two elements. The term “between” as used herein in reference to a first element being between a second element and a third element with respect to a direction means that the first element is closer to the second element as measured along the direction than the third element is to the second element as measured along the direction. The term “between” includes, but does not require that the first, second, and third elements be aligned along the direction.

Recitation of ranges of values herein are merely intended to serve as a shorthand method of referring individually to each separate value falling within the range including the stated ends of the range, unless otherwise indicated herein, and each separate value is incorporated into the specification as if it were individually recited herein.

Aspects of the disclosure will now be described in detail with reference to the drawings, wherein like reference numbers refer to like elements throughout, unless specified otherwise. Certain terminology is used in the following description for convenience only and is not limiting. The term “plurality,” as used herein, means more than one. The terms “a portion” and “at least a portion” of a structure include the entirety of the structure.

The headings and Abstract of the Disclosure provided herein are for convenience only and do not interpret the scope or meaning of the embodiments.

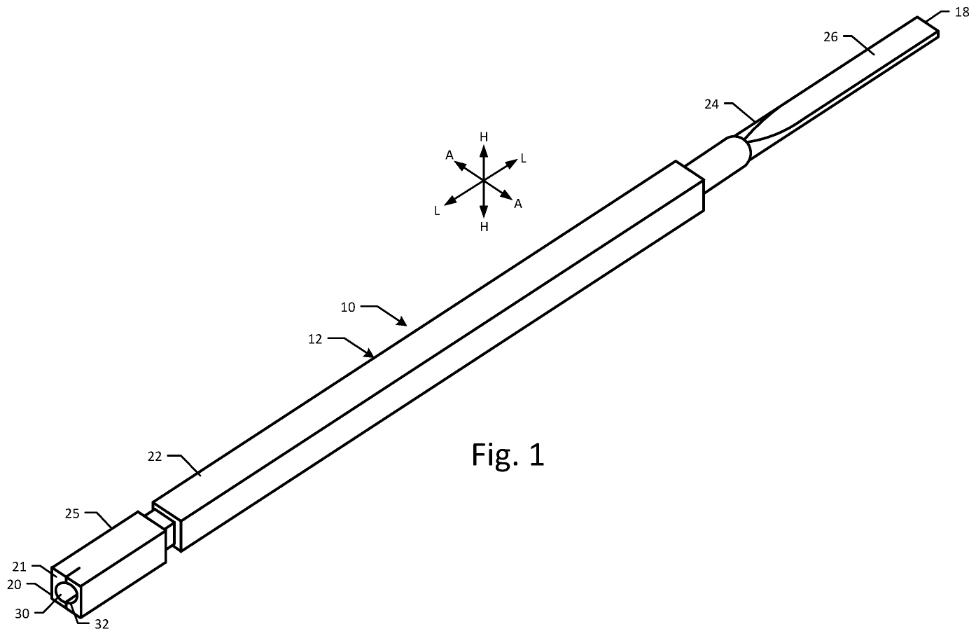

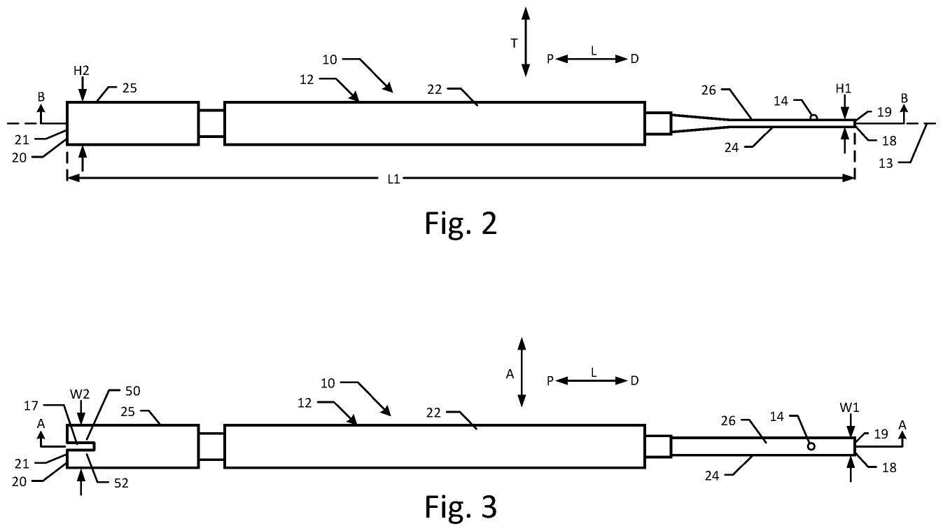

Referring to FIGS. 1 to 3, a specimen holder 10 may include a body 12 that carries a specimen 14, such as a biological materials and/or samples (e.g., eggs, sperm, and zygotes). The body 12 may be elongated along a direction, for example a longitudinal direction L, as shown in the illustrated embodiment. The body 12, may include a distal end 18 and a proximal end 20. As shown in the illustrated embodiment, the proximal end 20 may be opposite the distal end 18 with respect to the longitudinal direction L. According to one embodiment, the body 12 may extend in a distal component D of the longitudinal direction L from the proximal end 20 and terminate at the distal end 18, and the body 12 may extend in a proximal component P of the longitudinal direction L, opposite the distal component D, from the distal end 18 and terminate at the proximal end 20.

The body 12 may include a longitudinal axis 13 that is parallel to the longitudinal direction L. The longitudinal axis 13 may be a central longitudinal axis that lies in a midplane of the body 12. According to one embodiment, the body 12 may be symmetrical (e.g., radially symmetrical about the longitudinal axis 13.

The distal end 18 may include a surface 19 that faces in (e.g., is normal with respect to) the distal component D, as shown in the illustrated embodiment. The proximal end 20 may include a surface 21 that faces in (e.g., is normal with respect to) the proximal component P, as shown in the illustrated embodiment. The body 12 may include a length L1 measured from one of the distal end 18 and the proximal end 20 to the other of the distal end 18 and the proximal end 20 along the longitudinal direction L. The body 12 may be a monolithic (i.e., one-piece) structure, according to one embodiment.

The body 12 may include an outer surface 22 that extends from the distal end 18 to the proximal end 20. According to one embodiment, the outer surface 22 includes any surface of the body 12 that does not face toward any other surface of the body 12. The body 12 may include a distal portion 24 and a proximal portion 25. As shown, the distal portion 24 may carry the specimen 14 upon engagement of the body 12 with the specimen 14. The distal portion 24 may include and may extend from the distal end 18 towards the proximal portion 25. Similarly, the proximal portion 25 may include and extend from the proximal end 20 towards the distal portion 24.

According to one embodiment, the distal portion 24 may include up to a half of the length L1 of the body 12. Similarly, the proximal portion 25 may include up to a half of the length L1 of the body 12. Thus, according to one embodiment, the distal portion 24 and the proximal portion 25 may collectively include an entirety of the body 12. According to another embodiment, the body 12 may include additional portions (e.g., an intermediate portion) between the distal portion 24 and the proximal portion 25. According to one embodiment, the distal portion 24 and the proximal portion 25 may each include about 25% of the length L1 of the body 12, and the intermediate portion may include the remainder (about 50%) of the length L1 of the body 12.

The distal portion 24 may include a specimen surface 26 shaped to retain the specimen 14, for example against a force of gravity when the body 12 is arranged such that the longitudinal direction L is parallel to the force of gravity. The specimen surface 26 may be substantially planar, as shown in the illustrated embodiment. The specimen surface 26 may be curved, for example concave according to one embodiment. The specimen surface 26 may include texturing, grooves, or both. The specimen surface 26 may be a portion of the outer surface 22. Alternatively, the specimen surface 26 may be an internal surface, for example in an embodiment in which at least a portion of the body 12 is a tube.

The distal portion 24 may include a portion in the form of a stick, also referred to as a spatula. The spatula may include the specimen surface 26 in the form of a flat, planar surface. According to one embodiment, the distal portion 24 may include a portion in the form of a tube, also referred to as a straw. The straw may include the specimen surface 26 in the form of an internal surface that forms a cavity (e.g., that extends into the body 12 from the distal end 18.

As shown in the illustrated embodiment, the body 12 may include a width measured from one point on the outer surface 22 to another point on the outer surface 22 along a lateral direction A, which is perpendicular to the longitudinal direction L. The body 12 may include a height from one point on the outer surface 22 to another point on the outer surface 22 measured along a transverse direction T, which is perpendicular to both the longitudinal direction L and the lateral direction A.

According to one embodiment, the distal portion 24 may include a width W1 (e.g., measured at or proximate to the distal end 18). According to one embodiment, the distal portion 24 may include a height H1 (e.g., measured at or proximate to the distal end 18). The width W1 may be greater than the height H1, such that the distal portion 24 includes a rectangular cross-sectional shape within a plane that is normal to the longitudinal direction L. Alternatively, the width W1 may be equal to or less than the height H1.

The width may vary at different locations along the length L1 of the body 12. For example, the body 12 may have a minimum width. As shown, the minimum width may be the width W1 at the distal portion 24, for example at the distal end 18. The body 12 may have a maximum width W2 (e.g., at the proximal portion 25, for example at the proximal end 20). According to one embodiment, the distal portion 24 may include a constant width along the length of the distal portion 24. According to another embodiment, the width may taper along the distal portion 24 (e.g., getting smaller as the distal portion approaches the distal end 18).

The height may vary at different locations along the length L1 of the body 12. For example, the body 12 may have a minimum height. As shown, the minimum height may be the height H1 at the distal portion 24, for example at the distal end 18. The body 12 may have a maximum height H2 (e.g., at the proximal portion 25, for example at the proximal end 20). According to one embodiment, the distal portion 24 may include a constant height along the length of the distal portion 24. According to another embodiment, the height may taper along the distal portion 24 (e.g., getting smaller as the distal portion approaches the distal end 18).

According to one embodiment, the proximal portion 25 may be shaped such that the width of the proximal portion 25, for example the maximum width W2, is equal to the height of the proximal portion 25, for example the maximum height H2. According to another embodiment, the proximal portion 25 may be shaped such that the width of the proximal portion 25, for example the maximum width W2, is less than or greater than the height of the proximal portion 25, for example the maximum height H2.

Referring to FIGS. 1 to 12, the body 12 may include a cavity 30 (e.g., that extends into the proximal portion 25 from an opening 32. As shown, the opening 32 may be formed by the proximal end 20 (e.g., the surface 21). According to one embodiment, the cavity 30 may terminate within the proximal portion 25 (e.g., at a base surface 34). At least a portion of the cavity 30 may be formed by an inner surface 36 of the proximal portion 25. The inner surface 36 may form a cross-sectional shape of the cavity 30 within a plane that is normal to the longitudinal direction L. The proximal portion 25 may include an outer surface 38 (e.g., a portion of the outer surface 22) that forms a cross-sectional shape of the proximal portion 25 within a plane that is normal to the longitudinal direction L.

According to one embodiment, the cross-sectional shape of the proximal portion 25 may be different than the cross-sectional shape of the cavity 30 (e.g., the cross-sectional shape of the proximal portion 25 may be a square and the cross-sectional shape of the cavity 30 may be a circle). According to another embodiment, the cross-sectional shape of the proximal portion 25 may be the same as cross-sectional shape of the cavity 30.

As shown, a cavity length CL of the cavity 30 may extend along the longitudinal direction L (e.g., from the proximal end 20 into the proximal portion 25). The cavity length CL may include a maximum value when measured from the opening 32 to the base surface 34. The cavity 30 may include a cavity width CW that extends along the lateral direction A (e.g., from a first point on the inner surface 36 to a second point on the inner surface 36 that is opposite the first point). The cavity 30 may include a cavity height CH that extends along the transverse direction T.

Referring to FIGS. 1 to 16, the body 12 (e.g., the proximal portion 25) may include at least one slot 17. As shown, a slot length SL of the slot 17 may be measured along the longitudinal direction L (e.g., from the proximal end 20 into the proximal portion 25). The slot 17 may include a slot width SW that is measured along the lateral direction A (e.g., from the outer surface 38 to the inner surface 36). According to one embodiment, the slot 17 may extend in a straight line along the transverse direction T from the outer surface 38 to the inner surface 36. The slot 17 may include a slot height SH that extends along the transverse direction T from the outer surface 38 to the inner surface 36.

According to one aspect of the disclosure, the cavity length may be greater than the slot length SL, the cavity width CW may be greater than the slot width SW, the cavity height CH may be greater than the slot height SH, or any combination thereof.

According to one embodiment, the body 12 (e.g., the proximal portion 25) may be pliable. The cavity 30 may increase in size sufficiently such that a component (such as a wireless transponder) fits through the opening 32, even if the component is slightly larger than the opening, and a biasing force generated by the proximal portion 25 attempting to return to its unbiased configuration holds the component in place within the cavity. Upon removal of the component from the cavity 30, the proximal portion 25 may resiliently return to its original (e.g., unbiased) size.

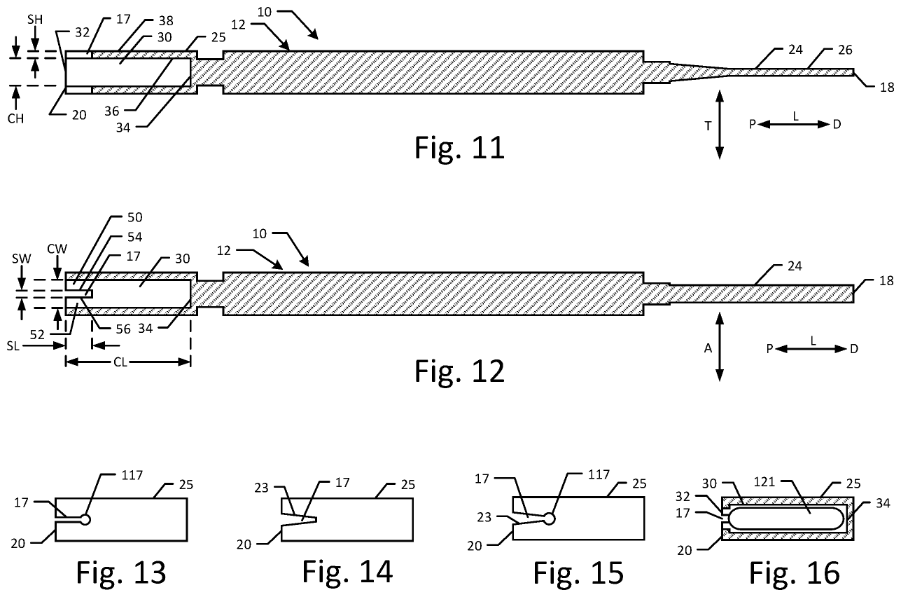

The at least one slot 17 may include different sizes and shapes. According to one embodiment, the slot 17 may be linear (e.g., a straight line or rectangular shape that extends along the longitudinal direction L) as shown in FIGS. 11 and 12. According to one embodiment, the slot 17 may include (e.g., may terminate at) an expanded portion 117 as shown in FIGS. 13 and 15. The expanded portion 117 may be rounded (e.g., circular) so as to reduce stresses when the slot 17 changes size. The expanded portion 117 may also prevent or reduce fractures or breakage (particularly at cold temperatures). According to one embodiment, the slot 17 may include a non-linear portion 23 (e.g., tapered) with a width SW that varies (e.g., decreases or increases along the longitudinal direction L) as shown in FIGS. 14 and 15. The slot 17 may include both the non-linear portion 23 and the expanded portion 117, as shown in FIG. 15.

The expanded portion 117 may function as a hinge about which the slot 17 changes shape. Thus, according to one embodiment, the expanded portion 117 may remain a constant size regardless of whether the cavity 30 is loaded or unloaded.

According to one embodiment, the opening 32 of the cavity 30 may be reduced in size (e.g., cross-sectional area) compared to a remainder of the cavity 30. Thus, the opening 32 may increase in size as a component 121 is inserted into the cavity 30, and upon clearing the opening 32, the proximal portion 25 may resiliently return to its original size with the component 121 trapped within the cavity 30 (e.g., between the reduced opening 32 and the base surface 34. Thus, the component 121 may be trapped without the need for the biasing force generated by the proximal portion 25 attempting to return to its unbiased configuration as shown in FIG. 16.

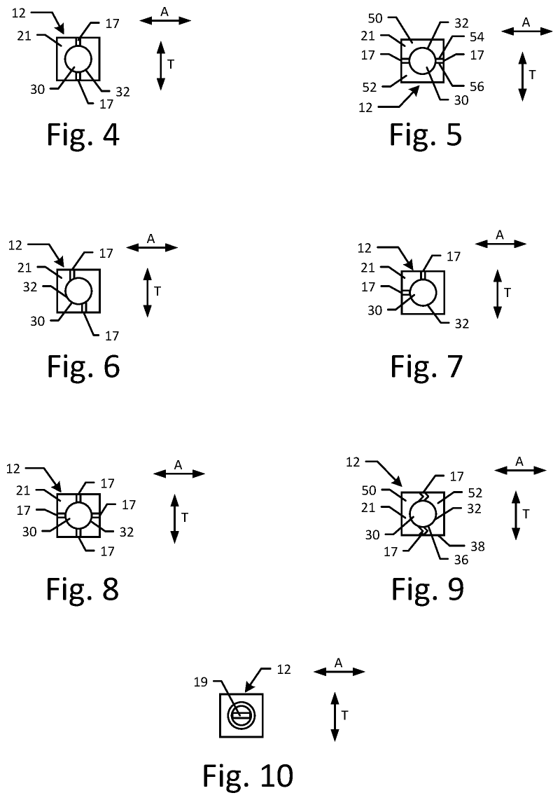

The at least one slot 17 may include first and second slots 17. The first and second slots 17 may be aligned such that a straight line that is perpendicular to the longitudinal direction L also intersects both of the first and second slots 17 (e.g., as shown in FIGS. 4 and 5). According to one embodiment, the aligned first and second slots 17 may be positioned in a midplane of the body 12 (e.g., a plane parallel to the longitudinal direction L about which the body 12 is symmetrical). Alternatively, one or more of the at least one slot 17 may be offset from the midplane. The at least one slot 17 may include slots 17 that are offset (i.e., not aligned) with one another (e.g., as shown in FIGS. 6 and 7). It will be appreciated by those of skill in the art that the at least one slot 17 may include other numbers of slots, such as four slots, (e.g., including two pairs of aligned slots as shown in FIG. 8).

The proximal portion 25 may include sub-portions (e.g., fingers) separated by the at least one slot 17. As shown the proximal portion 25 may include a first proximal portion 50 and a second proximal portion 52 separated by the first and second slots 17. The at least one slot 17 may have a shape formed by a first side surface 54 and a second side surface 56 that face one another.

According to one embodiment, the first side surface 54 may extend from the outer surface 38 (e.g., of the first proximal portion 50) to the inner surface 36 (e.g., of the first proximal portion 50), and the second side surface 56 may extend from the outer surface 38 (e.g., of the second proximal portion 52) to the inner surface 36 (e.g., of the second proximal portion 52).

The first and second side surfaces 54 and 56 may be planar (e.g., within a plane parallel to the longitudinal direction L as shown in FIGS. 4 to 8). The first and second side surfaces 54 and 56 may be non-planar (e.g., as shown in FIG. 9). The body 12 (e.g., the proximal portion 25) may include an unloaded configuration (e.g., when the cavity 30 is empty), and according to one embodiment, in the unloaded configuration the slot width SW may be zero (i.e., the first and second side surfaces 54 and 56 may be in contact with one another). According to one embodiment, in the unloaded configuration the slot width SW may be greater than zero (i.e., the first and second side surfaces 54 and 56 may be separated by a gap).

According to one aspect of the disclosure, the cavity 30 may include a maximum cross-sectional dimension (e.g., one of the cavity height CH and the cavity width CW), measured in a direction perpendicular to the longitudinal direction L. The proximal portion 25 may be resilient such that the maximum cross-sectional dimension increases from a first size when the body 12 is in the unloaded configuration (e.g., in an unbiased state) to a second size when the proximal portion 25 is in a loaded configuration (e.g., with a component positioned within the cavity 30). The loaded configuration may include a biased state, as described herein.

According to one embodiment, the cavity width CW, the cavity height CH, or both the cavity width CW and the cavity height CH may increase as the proximal portion transitions from the unloaded configuration to the loaded configuration. According to one embodiment, a maximum cross-sectional dimension of the distal end 18 (e.g., the width W1 or the height H1) may be less than the maximum cross-sectional dimension of the cavity 30 when the proximal portion 25 is in the loaded configuration.

In an embodiment in which the distal portion 24 includes a straw, the straw may be sized such that a maximum cross-sectional dimension of a cavity of the straw, measured in a direction perpendicular to the longitudinal direction L, may be less than the maximum cross-sectional dimension of the cavity 30.

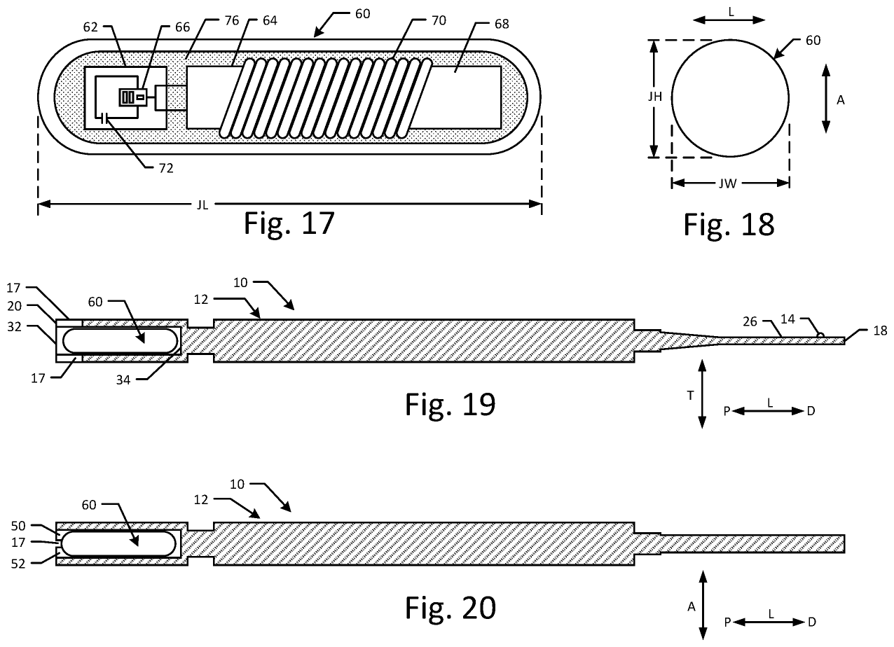

Referring to FIGS. 17 and 18, the specimen holder 10 may include a wireless transponder 60. The wireless transponder 60 may take a variety of forms. For example, the wireless transponder 60 may be in the form of an active, passive, or battery-assisted radio frequency identification transponders (RFID tags) that employs an integrated circuit to store and return a unique identifier. Active RFID transponders include a dedicated power source (e.g., a chemical battery cell) to power the RFID transponder. Passive RFID transponder do not include a dedicated power source, but rather derive power from an interrogation signal, typically charging a capacitor, which provides sufficient power to provide a return signal (e.g., back scatter signal) with unique identifying information imposed thereof. Battery-assisted RFID transponders generally detect an interrogation signal, but employ a dedicated power source (e.g., chemical battery cell) to primarily power the operations.

Also, for example, micro-electro-mechanical systems (MEMS) transponders employ one or typically more mechanical elements which mechanically vibrate or oscillate at respective frequencies to return a unique identifier. These MEMS transponders are mechanically based and typically do not employ integrated circuits, nor do they typically store unique identifiers in memory. The terms “integrated circuit RFID transponder” and “non-MEMS RFID transponder” are used herein to distinguish non-mechanical RFID transponders from mechanical or MEMS based transponders.

The wireless transponder 60, according to one embodiment, may be able to withstand cold temperatures (e.g., negative 150 degrees Celsius and below; negative 196 degrees Celsius and below) and continue to operate. In particular, the wireless transponder 60 may preferably be able to withstand multiple instances of temperature cycling between cold temperatures (e.g., negative 150 degrees Celsius below; negative 196 degrees Celsius below) and relatively warmer temperatures to which the containers may be exposed when removed from a cryogenic cooler or dewar. The wireless transponder 60 may advantageously take the form of passive wireless transponders, which rely on power from interrogation signals to provide responses, for example via backscattering. MEMS transponders may be particularly suitable for operation at cold temperatures.

The wireless transponder 60 may include a printed circuit board 62 and an antenna 64. The printed circuit board 62 may carry a transponder circuit 66 (e.g., radio, transmitter, backscatter circuit) communicatively coupled to the antenna 64. As shown, the antenna 64 may include a rod 68, (e.g., a ferrite rod) with a coil 70 wound around the rod 68. The wireless transponder 60 may include a power source 72 (e.g., capacitor, chemical battery). The wireless transponder 60 may include a capsule 74 that at least partially surrounds one or more of the components of the wireless transponder 60. An interior space within the capsule 74 may be occupied by a potting agent 76.

The wireless transponder 60 may have a length JL measured from one end of the wireless transponder 60 to an opposite end of the wireless transponder 60 along a direction of elongation of the wireless transponder 60. The wireless transponder 60 may have a width JW and a height JH measured perpendicular to one another, and perpendicular to the length JL.

Referring to FIGS. 11 to 20, the wireless transponder 60 may have a cylindrical cross-sectional shape. However, it will be appreciated that the shape of the wireless transponder 60 may vary, and may be selected so as to facilitate insertion into the cavity 30. As shown, a leading edge of the wireless transponder 60 may be tapered, or have rounded edges so as to limit interference during entry of the wireless transponder 60 into the cavity 30.

According to one aspect of the disclosure, a method of assembling the specimen holder 10 may include positioning the wireless transponder 60 adjacent the proximal end 20 of the body 12. The wireless transponder 60 may be inserted through the opening 32 and into the cavity 30. Inserting the wireless transponder 60 into the cavity 30 may increase a cross-sectional dimension of the cavity 30 (e.g., one or both of the cavity width CW and the cavity height CH). The increased cross-sectional dimension of the cavity 30 may be a result of a cross-sectional dimension of the wireless transponder 60 (e.g., one or both of the width JW and the height JH being slightly larger than the corresponding cross-sectional dimension of the cavity 30).

According to one embodiment, inserting the wireless transponder includes abutting both the first portion 50 and the second portion 52 with the wireless transponder 60. Abutting both the first portion 50 and the second portion 52 may increase the width SW of the at least one slot 17. Inserting the wireless transponder 60 through the opening may include positioning an entirety of the wireless transponder 60 within the cavity 30 such that entire length JL of the wireless transponder 60 is between the opening 32 and the base surface 34. In other words, in the loaded configuration, the specimen holder 10 may be devoid of a portion of the wireless transponder 60 that protrudes out of the cavity 30 through the opening 32. Securing the wireless transponder 60 within the cavity 30 may be via friction fit (e.g., due to the corresponding shapes of the wireless transponder 60 and the cavity 30).

According to one aspect of the disclosure a method of collecting a biological specimen (e.g., the biological specimen 14) may include any of the steps described above as part of the method of assembling the specimen holder 10. The method of collecting may include positioning the biological specimen 14 on a surface (e.g., the surface 26) of the body 12. As shown, the biological specimen 14 may be positioned closer to the distal end 18 than the biological specimen 14 is positioned from the proximal end 20.

The method of collecting may include collecting the biological specimen 14 from a patient. The method may include (e.g., after inserting the wireless transponder 60), interrogating the wireless transponder 60, and associating the collected biological specimen 14 and/or the patient with the specimen holder 10 upon which the biological specimen 14 is supported. According to one embodiment, the biological specimen 14 may be positioned on the surface 26 of the body 12 before the wireless transponder 60 is inserted through the opening 32 and into the cavity 30.

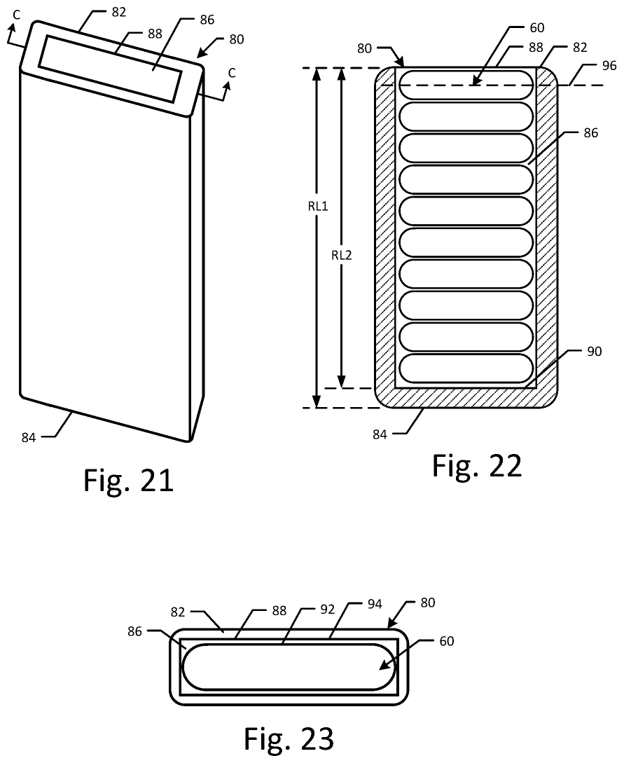

Referring to FIGS. 21 to 26, a cartridge 80 may include a first end 82 and a second end 84. The cartridge 80 may be elongate along a direction (e.g., a direction along which the first end 82 is opposite the second end 84). The cartridge 80 may include a first length RL1 measured along the direction from the first end 82 to the second end 84. The cartridge 80 may include a cavity 86 that extends from an opening 88 formed by the first end 82 toward the second end 84 (e.g., along the direction of elongation). The cavity 86 may include a second length RL2 measured along the direction from the opening 88 to a base surface 90 of the cartridge 80. As shown, the base surface 90 may be opposite the second end 84 (e.g., the second end 84 may be an external or outer surface and the base surface 90 may be an inner surface). The base surface 90 may be positioned closer to the second end 84 than the base surface 90 is from the first end 82.

The cartridge 80 may include a plurality of wireless transponders (e.g., a plurality of the wireless transponders 60) positioned within the cavity 86, each of the plurality of wireless transponders 60 may include an outer peripheral shape 92 that corresponds to a cross-sectional shape 94 of the cavity 86. As shown, the outer peripheral shape 92 and the cross-sectional shape 94 may both be within a plane perpendicular to the direction of elongation of the cartridge 80. According to one embodiment, the outer peripheral shape 92 may correspond to the cross-sectional shape 94 such that relative movement of the plurality of wireless transponders 60 and the cartridge 80 is constrained in all degrees of freedom other than translation along the direction of elongation.

The outer peripheral shape 92 and the cross-sectional shape 94 may be different shapes (e.g., one being rectangular and the other being elliptical/pill-shaped). The outer peripheral shape 92 and the cross-sectional shape 94 may correspond such that one or more (e.g., two) dimensions of the outer peripheral shape 92 are slightly smaller than corresponding dimensions of the cross-sectional shape 94. The plurality of wireless transponders 60 may be stacked within the cavity 86 (e.g., such that the plurality of wireless transponders 60 are aligned along the direction of elongation) such that translation in any direction perpendicular to the direction of elongation is blocked (e.g., with some limited movement possible based on manufacturing tolerances).

According to one aspect of the disclosure, the plurality of wireless transponders 60 may be elongate along an axis 96, and the plurality of wireless transponders 60 may be positioned within the cavity 86 such that the axis 96 of each of the plurality of wireless transponders 60 is perpendicular to the direction of elongation of the cartridge 80. As shown in the illustrated embodiment, the plurality of wireless transponders 60 may be positioned within the cavity 86 such that the axis 96 of each of the plurality of wireless transponders 60 lies within a plane that is parallel to the direction of elongation of the cartridge 80.

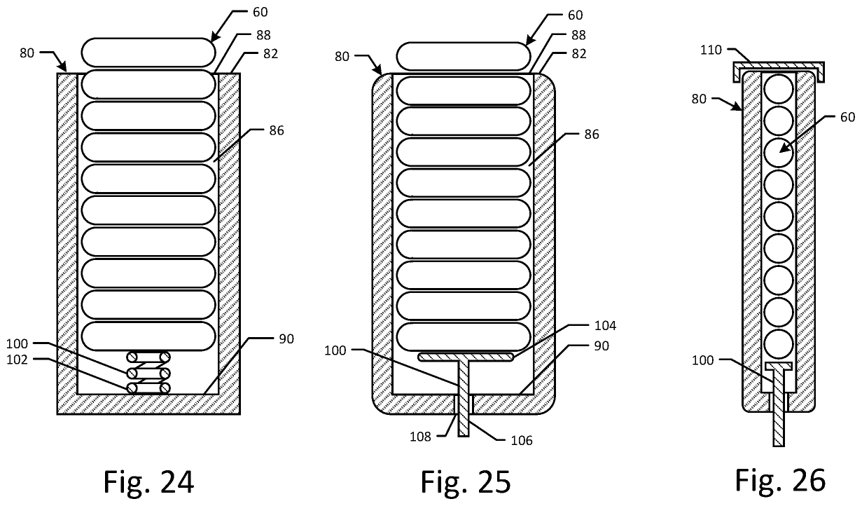

The cartridge 80 may include an actuator 100 positioned at least partially within the cavity 86 such that the movement of the actuator 100 translates the plurality of wireless transponders 60 relative to the cartridge 80 along the direction of elongation. The actuator 100 may include a biasing member 102 (e.g., a spring) that exerts a force on the plurality of wireless transponders 60 directed towards the opening 88 as shown in FIG. 24. The actuator 100 may include a first portion 104 positioned within the cavity 86 (e.g., that abuts one of the plurality of wireless transponders 60) and a second portion 106 that extends through an opening 108 in the second end 84 and is positioned outside the cavity 86 as shown in FIGS. 25 and 26.

The cartridge 80 may include a stopper 110 secured to the cartridge 80 that blocks at least a portion of the opening 88, thereby blocking movement of the plurality of wireless transponders 60 through the opening 88 and out of the cavity 86. The stopper 110 may be removable from the cartridge 80 such that the stopper 110 no longer blocks any portion of the opening 88. According to one aspect of the disclosure, the stopper 110 may be in the form of a cap or fitting that attachable and removable (e.g., via a friction fit, snap fit, threaded connection, etc.). According to one aspect of the disclosure, the stopper 110 may be in the form of an adhesive strip (e.g., tape, a pull tab, etc.).

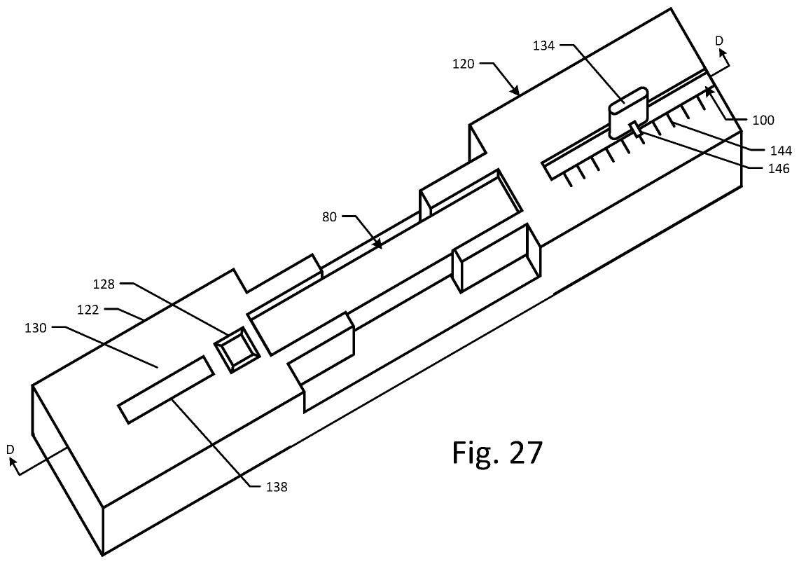

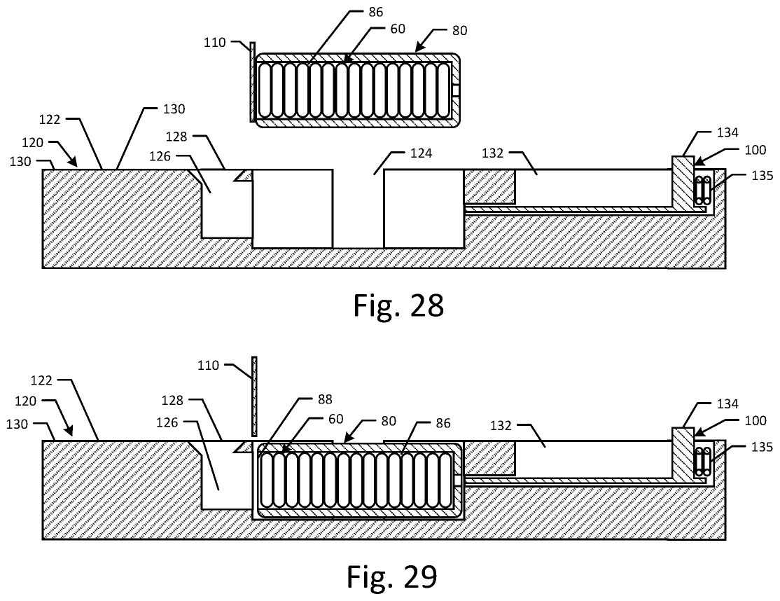

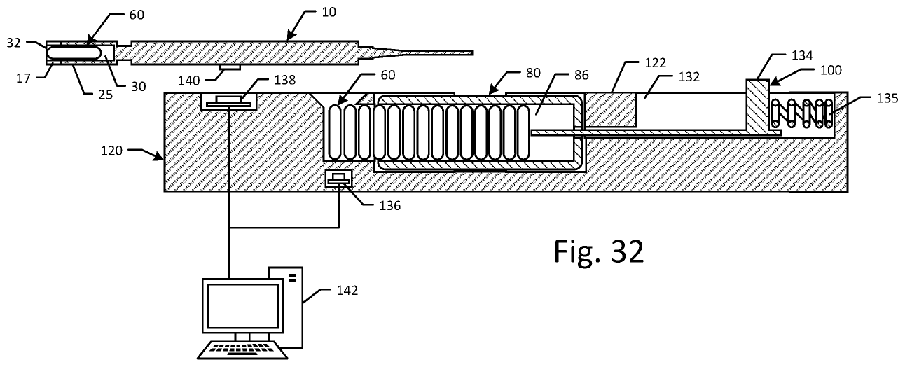

Referring to FIGS. 27 to 32, a wireless transponder dispenser 120 may include a housing 122 that includes a recess 124. The recess 124 may be shaped to receive a cartridge (e.g., the cartridge 80). The housing 122 may include a housing cavity 126 that corresponds in shape to the cavity 86 of the cartridge 80. As shown, the cartridge 80 may be receivable within the recess 124 of the housing 122 such that the cavity 86 of the cartridge 80 is aligned with the housing cavity 126.

The housing 122 may include an opening 128 formed by an outer surface 130 of the housing 122. As shown, the opening 128 may provide passage into the housing cavity 126 along a path that is angularly offset to the direction of elongation of the cartridge 80 when the cartridge 80 is positioned within the recess 124 (as shown in FIG. 29).

Referring to FIGS. 1 to 32, a method of use of the wireless transponder dispenser 120 may include a method of dispensing at least one wireless transponder. The method may include positioning the cartridge 80 within the recess 124 of the housing 122 such that the cavity 86 of the cartridge 80 is aligned with the housing cavity 126, which extends into the housing 122 and away from the cartridge 80 (e.g., as shown in FIG. 29). After the cartridge is positioned within the recess 124, the method may include removing the stopper 110. After the cartridge is positioned within the recess 124, the method may include moving at least one of the wireless transponders 60 from the cavity 86 of the cartridge 80, through the opening 88, and into the housing cavity 126.

According to one embodiment, moving the at least one wireless transponder 60 may include translating the at least one wireless transponder 60 relative to the cartridge 80 along a first direction (e.g., the direction of elongation of the cartridge 80). While translating the at least one wireless transponder 60 relative to the cartridge 80 along the first direction, the method may include restricting relative movement of the at least one wireless transponder 60 and the cartridge 80 in all other degrees of freedom.

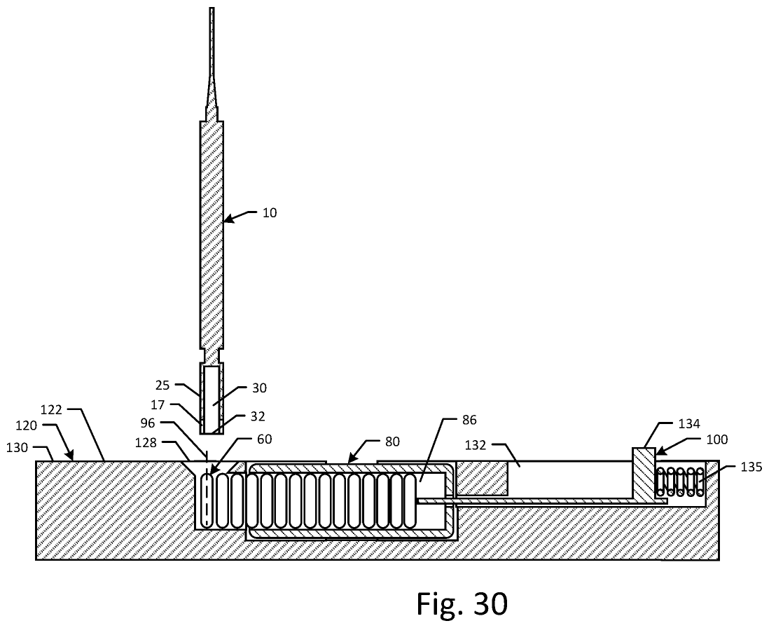

The method may include aligning one of the wireless transponders 60 with the opening 128 in the housing 122 (e.g., as shown in FIG. 30). The method may include inserting a portion of a specimen holder (e.g., the proximal portion 25 of the specimen holder 10) through the opening 128 in the housing 122 and into the housing cavity 126 such that the one of the wireless transponders 60 is aligned with a cavity (e.g., the cavity 30) of the specimen holder 10 formed in the proximal portion 25.

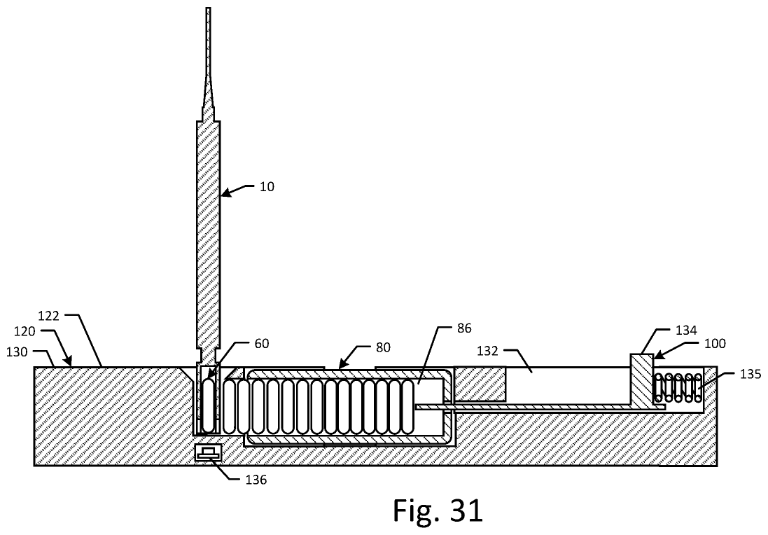

The method may include moving the specimen holder 10 (e.g., translating the specimen holder 10 along a direction that is parallel to the axis 96) relative to the aligned wireless transponder 60, thereby inserting the aligned wireless transponder 60 through an opening (e.g., the opening 32) in the specimen holder 10 and into the cavity 30 (e.g., as shown in FIG. 31). The at least one wireless transponder 60 may be elongate along the axis 96, and positioning the cartridge 80 within the recess 124 may include orienting the at least one wireless transponder 60 such that the respective axis 96 of each of the at least one wireless transponders 60 is parallel to the second direction. Translating the specimen holder 10 relative to the aligned wireless transponder 60 may include moving the specimen holder 10 along a second direction that is perpendicular to the first direction.

The wireless transponder dispenser 120 may include an actuator (e.g., the actuator 100). The actuator 100 may be supported by the housing 122 (e.g., within a slot 132 that restricts movement other than translation along the first direction). Moving the at least one wireless transponder 60 from the cavity 86 of the cartridge 80, through the opening 88, and into the housing cavity 126 may include actuating the actuator 100. The actuator 100 may be manually operated, such that actuating the actuator 100 requires physical input from a user (e.g., imparting a force on a handle 134 of the actuator 100 when movement of the at least one wireless transponder 60 is desired). The actuator 100 may include a biasing member 135 that exerts a force in the first direction and thereby automatically moves the at least one wireless transponder 60 in the first direction whenever there is space within the housing cavity 126 to receive one of the wireless transponders 60 (e.g., after removal of one of the wireless transponders 60 through the opening 128).

According to one embodiment, the wireless transponder dispenser 120 may be devoid of an actuator that moves the wireless transponders 60 within the cartridge 80 and the housing 122. For example, the housing 122 may be mounted vertically, such that the wireless transponders 60 fall via the force of gravity into alignment with the opening 128.

Inserting the aligned wireless transponder 60 through the opening 32 and into the cavity 30 may include increasing a dimension (e.g., the cavity height CH, the cavity width CW, or both) of the cavity 30. Increasing the dimension of the cavity 30 may include increasing a dimension (e.g., the slot height SH, the slot width SW, or both) of the at least one slot 17. The method may include securing the aligned wireless transponder 60 within the cavity 30 via a friction fit.

The wireless transponder dispenser 120 may include a wireless interrogator 136. The wireless interrogator 136 may be supported by the housing 122 (e.g., positioned such that the aligned wireless transponder 60 is aligned with a beam axis of the wireless interrogator 136. The wireless interrogator 136 may be positioned such that only the aligned wireless transponder 60 is interrogated by the wireless interrogator 136, while the remaining wireless transponders 60 are not interrogated by the wireless interrogator 136. The wireless transponder dispenser 120 may include an optical scanner 138. The optical scanner 138 may be supported by the housing 122. According to one embodiment, the optical scanner 138 may be positioned so as to scan a label 140 on the specimen holder 10 when the specimen holder 10 is removed from the housing cavity 126. According to one embodiment, the optical scanner 138 may be positioned so as to scan the label 140 on the specimen holder 10 while the proximal portion 25 is positioned within the housing cavity 126.

The method may include associating the wireless transponder 60 positioned within the cavity 30 of the specimen holder 10 with the specimen holder 10, with the biological specimen 14 carried by the specimen holder 10, or with both the specimen holder 10 and the biological specimen 14 carried by the specimen holder 10. Associating the wireless transponder 60 may include interrogating the wireless transponder 60 after it is positioned within the cavity 30 of the specimen holder 10 (e.g., while the proximal portion 25 is positioned within the housing cavity 126). Associating the wireless transponder 60 may include scanning the label 140 (e.g., with the optical scanner 138). One or both of the wireless interrogator 136 and the optical scanner 138 may be communicatively connected (e.g., wired, wirelessly) to a network, database, user interface 142, or any combination thereof.

The label 140 may include information that identifies the specimen holder 10, the biological specimen 14 supported by the specimen holder 10, the patient from which the biological specimen 14 was taken, etc. The label 140 may include machine readable symbols (e.g., bar codes, QR codes, etc.). Interrogating the wireless transponder 60 may uniquely identify the specific wireless transponder 60 from other wireless transponders. Scanning the label 140 may uniquely identify the specific specimen holder 10 from other specimen holders. The network, database, user interface 142 may associate the uniquely identified wireless transponder 60 with the uniquely identified specimen holder 10. The association may enable the wireless transponder 60 to be interrogated, thereby also recalling the information from the label 140 without needing to optically scan or read the label 140.

The method may include withdrawing the specimen holder 10 and the wireless transponder 60, secured within the cavity 30, through the opening 128 in the housing 122. The method may include moving a second wireless transponder 60 from the cavity 86, through the opening 88, and into the housing cavity 126. After withdrawing the specimen holder 10 and the wireless transponder 60 captured within the cavity 30, the method may include aligning the second wireless transponder 60 with the opening 128 in the housing 122.

The wireless transponder dispenser 120 may include visual indicators 144 that identify the number of wireless transponders 60 supported by the wireless transponder dispenser 120 and available for insertion into respective specimen holders 10. According to one embodiment, the visual indicators 144 may include numbers on an exterior of the housing 122. The numbers may be adjacent the handle 134, such that after the actuator 100 moves to advance the wireless transponders 60, a portion 146 of the handle 134 is aligned with one of the numbers, and that number indicates how many of the wireless transponders 60 remain within the wireless transponder dispenser 120.

The above description of illustrated embodiments, including what is described in the Abstract, is not intended to be exhaustive or to limit the embodiments to the precise forms disclosed. Although specific embodiments of and examples are described herein for illustrative purposes, various equivalent modifications can be made without departing from the spirit and scope of the disclosure, as will be recognized by those skilled in the relevant art. The teachings provided herein of the various embodiments can be applied to specimen holders, cartridges, and wireless transponder dispensers, not necessarily the exemplary specimen holders, cartridges, and wireless transponder dispensers generally described above.

Many of the methods described herein can be performed with variations. For example, many of the methods may include additional acts, omit some acts, and/or perform acts in a different order than as illustrated or described.

The various embodiments described above can be combined to provide further embodiments. To the extent that they are not inconsistent with the specific teachings and definitions herein, all of the U.S. patents, U.S. patent application publications, U.S. patent applications, foreign patents, foreign patent applications and non-patent publications referred to in this specification and/or listed in the Application Data Sheet, including but not limited to PCT Pub. No. WO 2021/097330, filed Nov. 13, 2020; U.S. application Ser. No. 17/321,174, filed May 14, 2021; U.S. application Ser. No. 17/483,603, filed Sep. 23, 2021; and U.S. application Ser. No. 17/547,094, filed Dec. 9, 2021 are incorporated herein by reference, in their entirety. Aspects of the embodiments can be modified, if necessary, to employ systems, circuits and concepts of the various patents, applications and publications to provide yet further embodiments.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

1. A specimen holder comprising:
a body elongated along a body longitudinal axis, the body having a distal end and a proximal end, the proximal end opposite the distal end with respect to the body longitudinal axis, the body having a maximum length measured from the proximal end to the distal end along a first direction that is parallel to the longitudinal axis, the body including a distal portion that includes the distal end and a proximal portion that includes the proximal end, the distal portion including a surface that carries a specimen upon engagement of the body with the specimen;
a cavity that extends into the proximal portion from an opening formed in the proximal end, the cavity terminating within the proximal portion, wherein at least a portion of the cavity is formed by an inner surface of the proximal portion; and
at least one slot that extends from the proximal end in the first direction, the at least one slot extends from an outer surface of the proximal portion to the inner surface.

2. The specimen holder of claim 1 wherein the cavity includes a cavity maximum length measured in the first direction, the at least one slot includes a slot maximum length measured along the first direction, and the cavity maximum length is greater than the slot maximum length.

3. The specimen holder of claim 2 wherein the at least one slot has a slot depth measured from the outer surface to the inner surface along a second direction that is perpendicular to the body longitudinal axis, the cavity includes a cavity depth measured in the second direction, and the cavity depth is greater than the slot depth.

4. The specimen holder of claim 3 wherein the at least one slot extends from the outer surface to the inner surface along the second direction.

5. The specimen holder of claim 1 wherein the at least one slot includes at least a first slot and a second slot.

6. The specimen holder of claim 5 wherein the first slot is aligned with the second slot such that a straight line that perpendicularly intersects the body longitudinal axis also intersects both the first slot and the second slot.

7. The specimen holder of claim 1 wherein the cavity includes a maximum cross-sectional dimension measured in a direction perpendicular to the longitudinal body axis, and the proximal portion is pliable such that the maximum cross-sectional dimension increases from a first size when the proximal portion is in an unbiased state to a second size when the proximal portion is in a biased state.

8. The specimen holder of claim 7 wherein the at least one slot includes a width, and at least a portion of the width increases when the proximal portion transitions from the unbiased state to the biased state.

9. The specimen holder of claim 7 wherein the distal end includes a maximum cross-sectional dimension measured perpendicular to the body longitudinal axis and parallel to the surface, and the maximum cross-sectional dimension of the distal end is less than the maximum cross-sectional dimension of the cavity when the proximal portion is in the unbiased state.

10. The specimen holder of claim 9 wherein the distal portion includes a spatula, the surface is a flat surface having a length measured from the distal end along the first direction, the cavity includes a cavity maximum length measured along the first direction, and the length of the flat surface is greater than the cavity maximum length.

11. The specimen holder of claim 1 wherein the cavity is a proximal cavity, the distal portion includes a straw, the surface is an internal surface that forms a distal cavity of the straw, the distal cavity extends into the distal portion from an opening formed in the distal end.

12. The specimen holder of claim 11 wherein the proximal cavity includes a first maximum cross-sectional dimension measured in a direction perpendicular to the longitudinal body axis, the distal cavity includes a second maximum cross-sectional dimension measured in the direction perpendicular to the longitudinal body axis, and the first maximum cross-sectional dimension is greater than the second maximum cross-sectional dimension.

13. The specimen holder of claim 1, further comprising a wireless transponder positioned within the cavity.

14. The specimen holder of claim 13 wherein the wireless transponder is held within the cavity by a friction fit.

15. The specimen holder of claim 1 wherein the body is a monolithic, one-piece member.

16. A method of assembling a specimen holder, the method comprising:
positioning a wireless transponder adjacent a distal end of a body, the body elongated along a body longitudinal axis, the body having a distal end and the body having a proximal end opposite the distal end with respect to the body longitudinal axis;
inserting the wireless transponder through an opening formed in the proximal end and into a cavity that extends into the proximal portion from the opening in the proximal end;
causing a cross-sectional dimension of the cavity that is measured in a direction perpendicular to the body longitudinal axis to increase while inserting the wireless transponder; and
while inserting the wireless transponder, abutting both a first portion of the proximal portion and a second portion of the proximal portion with the wireless transponder, the first portion and the second portion separated by at least one slot of the body, the slot including a length that extends from the proximal end in a direction parallel to the body longitudinal axis, the at least one slot further including a depth that extends from an inner surface of the proximal portion that forms the cavity to an outer surface of the proximal portion that faces away from the cavity,
wherein abutting both the first portion of the proximal portion and the second portion of the proximal portion with the wireless transponder increases a width of the at least one slot, the width extending from a first side surface of the first portion to a second side surface of the second portion, the first and second side surfaces each extending between the inner surface and the outer surface of the proximal portion.

17. The method of claim 16 wherein inserting the wireless transponder through the opening includes positioning an entirety of the wireless transponder within the cavity such that the specimen holder is devoid of a portion of the wireless transponder that protrudes out of the cavity through the opening.

18. The method of claim 16, further comprising:
securing the wireless transponder within the cavity via friction fit.

19. A method of collecting a biological specimen, the method comprising:
positioning a biological specimen on a surface of a body, the body elongated along a body longitudinal axis, and the body having a distal end and a proximal end opposite one another with respect to the body longitudinal axis, wherein the biological specimen is positioned closer to the distal end than the biological specimen is positioned from the proximal end;
inserting a wireless transponder through an opening formed in the proximal end and into a cavity that extends into the body from the opening;
causing a cross-sectional dimension of the cavity that is measured in a direction perpendicular to the body longitudinal axis to increase while inserting the wireless transponder; and
while inserting the wireless transponder, abutting both a first portion of the proximal portion and a second portion of the proximal portion with the wireless transponder, the first portion and the second portion separated by at least one slot of the body, the slot including a length that extends from the proximal end in a direction parallel to the body longitudinal axis, the at least one slot further including a depth that extends from an inner surface of the proximal portion that forms the cavity to an outer surface of the proximal portion that faces away from the cavity,
wherein abutting both the first portion of the proximal portion and the second portion of the proximal portion with the wireless transponder increases a width of the at least one slot, the width extending from a first side surface of the first portion to a second side surface of the second portion, the first and second side surfaces each extending between the inner surface and the outer surface of the proximal portion.

20. The method of claim 19, further comprising:
collecting the biological specimen from a patient; and
after the inserting the wireless transponder, reading the wireless transponder thereby associating the collected biological specimen with the patient.

21. The method of claim 19 wherein the biological specimen is positioned on the surface of the body before the wireless transponder is inserted through the opening.

22-40. (canceled)
